(19) United States
(12) Patent Application Publication
(10) Pub. No.: US 2020/0368135 A1
(43) Pub. Date: Nov. 26, 2020

(54) USE OF NON-DISSOLVED STARCH BASED PARTICLES

(71) Applicant: SPEXIMO AB, Lund (SE)

(72) Inventors: Malin SJÖÖ, Kävlinge (SE); Abdullah ALI, Skurup (SE); Jasmine BEDI SVENSSON, Solna (SE)

(21) Appl. No.: 16/966,351

(22) PCT Filed: Feb. 1, 2019
(86) PCT No.: PCT/SE2019/050090

§ 371 Date: Jul. 30, 2020

(30) Foreign Application Priority Data
Feb. 2, 2018 (SE) 1850124-7

(51) Int. Cl. A61K 8/73; A61K 8/02; A61K 8/92; A61Q 19/00; A61Q 1/12; C08B 30/02; A61K 8/06

(52) U.S. Cl. CPC A61K 8/732; A61K 8/0241; A61K 8/922; A61Q 19/00; A61K 2800/413; C08B 30/02; A61K 8/062; A61K 8/064; A61K 2800/10; A61Q 1/12

(57) ABSTRACT

The present invention relates to the use of non-dissolved starch based particles, having a particle size of 0.2-4 pm and having a composition of 0.3-5% by weight of protein, 0.1-4% by weight of lipids, 0-1.6% by weight fibre and <0.45% by weight ash and the remaining part of the composition is carbohydrate adding up to 100% by weight, in cosmetic formulations and the preparation of said particle.

### TECHNICAL FIELD OF THE INVENTION

The present invention relates to use of non-dissolved starch based particles, having a particle size of 0.2-4 μm and having a composition of 0.5-5% by weight of protein, 0.1-4% by weight of lipids, 0-1.6% by weight fibre and <0.45% by weight ash and the remaining part of the composition is carbohydrate adding up to 100% by weight, for providing a cosmetic formulation comprising oil and/or water and said starch particles. The invention further relates to a process for the preparation of said starch based particles.

### BACKGROUND ART

WO2012/082065 discloses particle stabilized emulsions or foams comprising two phases, such as oil and water, and solid starch particles having a granular size of 0.2-20 μm which are situated at the interface between the two phases for stabilization of said emulsions or foams. Not all emulsions as disclosed in this document are suitable for being used in cosmetic formulations.

U.S. Pat. No. 5,423,281 discloses cosmetic powders utilizing small granule starch having a size of less than 5 microns. These granules may be substituted for binder excipients in tableting compositions or the talc or granular starch ingredients of cosmetics/dusting powder compositions. The starches may be isolated from seeds by wet milling in the ordinary way as starch is produced commercially from potatoes or corn. The seeds are steeped and disintegrated by grinding or by high speed shearing. The starch is separated from the aqueous phase following removal of fibre by screening.

U.S. Pat. No. 7,563,473 discloses a quinoa protein concentrate containing at least about 50% by weight protein and a method for producing such a concentrate by milling quinoa fruit, separating embryo-rich fraction, extraction of oil to produce defatted quinoa, extraction of protein from defatted quinoa and separation of protein from the insoluble fibre of the defatted quinoa and drying the protein.

Khan Nadiya Jan et al, Structural, thermal and rheological properties of starches isolated from Indian quinoa varieties, International Journal of Biological Macromolecules 102 (2017), 315-322, discloses physico-chemical properties of native starches from Indian quinoa varieties. However, there is no disclosures of its use in cosmetic formulations in this publication.

Zhu et al, Quinoa starch: Structure, properties and applications, Carbohydrate Polymers 181 (2018) 851-861, discloses isolation, composition, granular and molecular structures, modifications and applications of quinoa starch.

The use of starch particles from certain botanical origins in different formulations such as emulsions or cosmetic products, such as creams and lotions, for use on different parts of the body sometimes creates an odour or smell which is undesirable. The separation and purification of small granular starches, <4 μm, can be challenging compared to larger starches due to the smaller particle size.

Furthermore, the use of starch particles in cosmetic products, such as in creams and lotions, need to have a homogenous structure with a good skin-feel.

There is a need within the art of especially green (i.e. natural and/or sustainable) cosmetic products where the use of chemicals is to be minimized or preferably omitted fully to provide such green cosmetic products without said drawbacks.

The inventors of the present invention, have found a solution to the above mentioned problems.

### SUMMARY OF THE INVENTION

In one aspect of the invention, there is provided use of non-dissolved starch based particles having a particle size of 0.2-4 μm and having a composition of 0.3-5% by weight of protein, 0.1-4% by weight of lipids, 0-1.6% by weight fibre and <0.45% by weight ash and the remaining part of the composition is carbohydrate adding up to 100% by weight for providing a cosmetic formulation comprising oil and/or water and said starch particles.

In another aspect, there is provided use of non-dissolved starch based particles having a particle size of 0.2-4 μm and having a composition of 0.3-5% by weight of protein, 0.1-4% by weight of lipids, 0-1.6% by weight fibre and <0.45% by weight ash and the remaining part of the composition is carbohydrate adding up to 100% by weight for providing a cosmetic formulation comprising oil and/or water and said starch particles, said starch particles providing solid surfaces in the cosmetic formulation.

In yet another aspect, there is provided a cosmetic formulation comprising above mentioned starch based particles and oil and/or water, wherein said starch particles are present in said oil or said starch particles are present in said water or said starch particles are present in both said water and oil.

In yet another aspect, there is provided a process for the preparation of said starch particle, comprising the following steps:
subjecting dehulled grains or grain press cake to wet or dry milling for providing a flour with the size of 0.2-2.0 mm;
mixing said flour with water for hydration during a period of 0.5-6 hours for efficient fibre separation;
separation of a light phase comprising mainly starch and protein and a heavy phase comprising fibre;
pH adjustment of light phase to pH 6-12;
separation of proteins and starch from light phase based on density and size; and
drying

### DETAILED DESCRIPTION OF THE DRAWINGS

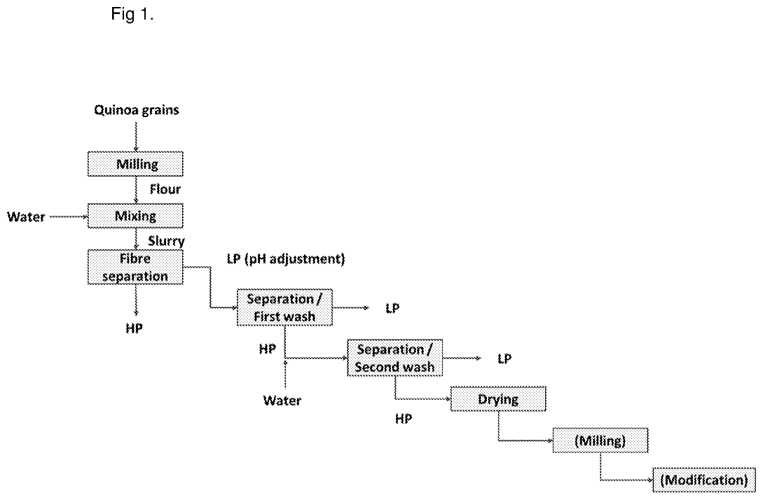

FIG. 1 discloses quinoa starch based particle production according to the present invention.

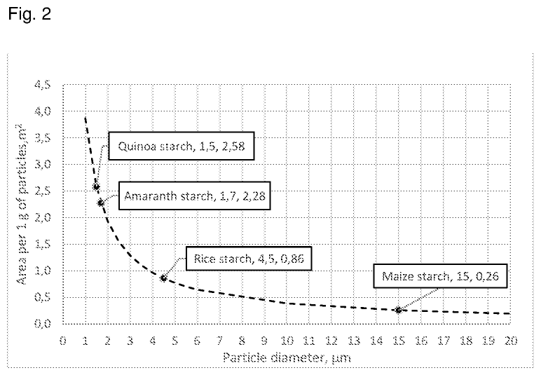

FIG. 2 discloses area per 1 g of particles for starch particles of different diameter. Labels indicate starch source, diameter (μm), Area (m²).

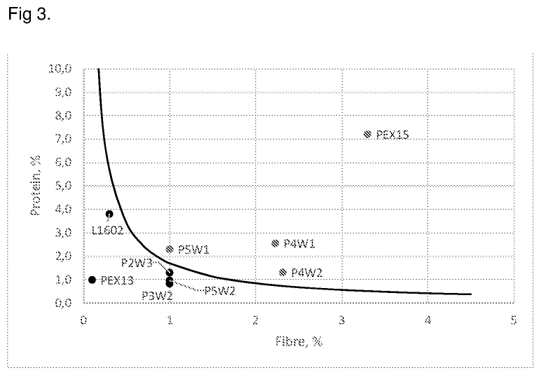

FIG. 3 discloses protein content (%) versus fibre content (%) for quinoa starch samples. The solid line is given by the identified relation between protein and fibre, i.e. Protein=1.7/Fibre.

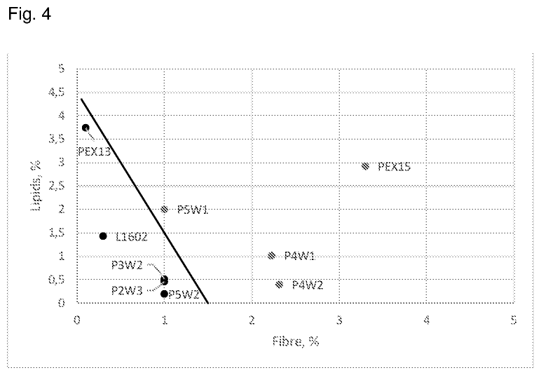

FIG. 4 discloses lipid content (%) versus fibre content (%) for quinoa starch samples. The solid line is given by the identified relation between lipids and fibre, i.e. Lipid=−3*Fibre+4.5.

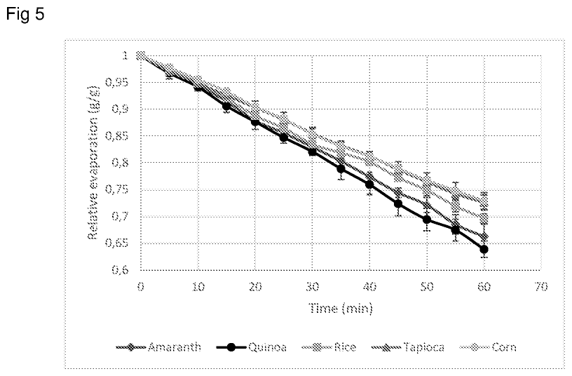

FIG. 5 discloses evaporation of excess water measured as relative evaporation with time for samples with differently sized starch particles for Experiment 1.

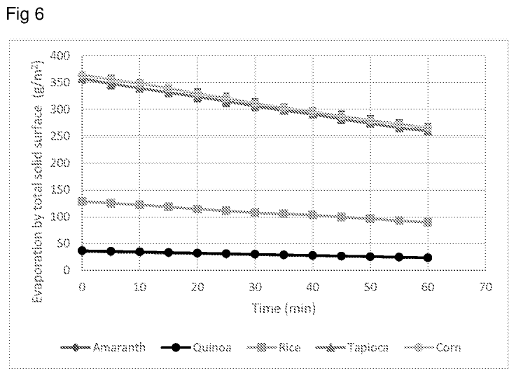

FIG. 6 discloses evaporation of excess water expressed as evaporation by solid surface with time for samples with differently sized starch particles for Experiment 10.

### DETAILED DESCRIPTION OF THE INVENTION

As stated above there has been provided use of non-dissolved starch based particles in cosmetic formulations, said starch particles having a particle size of 0.2-4 μm, e.g. 0.5-1 or 1-2 μm, and having a composition of 0.3-5% by weight of protein, about 0.5, 1, 2, 3, 4 or 5% by weight, 0.1-4% by weight of lipids, e.g. about 0.5, 1, 2, 2.5, 3, 3.5 or 4% by weight, 0-1.6% by weight fibre, e.g. about 0.2-1.5, about 0.4-0.8% by weight and <0.45% by weight ash and the remaining part of the composition is carbohydrate adding up to 100% by weight (calculated by dry weight). The carbohydrate is composed of amylose and amylopectin, but traces of other sugar components may be present. The specific composition of protein, lipid, fibre, and ash of said starch particles have been shown in the examples to provide desirable beneficial effects in the final cosmetic formulation. Starch is a natural based and renewable resource of raw material. The advantages of said starch particle is that it has been optimized for use in cosmetic formulations comprising oil and/or water where there is a desire to exclude or minimize chemical additives and/or ingredients from non-renewable resources. By using said starch particles in such cosmetic formulations, texture in the formulation and on skin is improved by the added solid surface, and undesired odour is at least reduced or omitted fully at the same time as the use of chemical additives and/or non-renewable are omitted. A desirable emulsion or suspension consistency is also obtained. In another embodiment, there is provided a non-dissolved starch based particle having a composition of 0.4-2% by weight of protein, e.g. 0.7-1.1, 0.1-1% by weight of lipids, e.g. 0.15-0.6, 0.1-1.2% by weight fibre, e.g. 0.8-1.1, and <0.25% by weight ash and the remaining part of the composition is carbohydrate adding up to 100% by weight. In an embodiment of the invention the composition of the starch based particle is within the limitation of the equations Protein≤1.7/Fibre and Lipid≤−3*Fibre+4.5.

The starch particles may be non-gelatinized or technically, such as dry heat treatment, or chemically modified such as OSA (octenyl succinic anhydride) modified. The modification may adjust surface properties.

The starch particles according to the invention is from a botanical source chosen from quinoa, amaranth, tapioca, rice, oat, wheat, barley, millet, canihua, including waxy and high amylose varieties of any of the previously mentioned botanical sources. The starch particle may also be obtained from another botanical source not mentioned here as long as the starch particle has a size in the range of 0.2-4 μm, e.g. 0.5, 1, 1.5, 2, 2.5, 3, 3.5 μm. In an embodiment, there is provided use of said starch particles, for providing a cosmetic formulation comprising oil and/or water, said starch particles providing solid surfaces in the cosmetic formulation, wherein the starch particles providing solid surfaces contribute to an area of at least 1 m² per g of particles when added to a formulation. Solid surfaces contribute to the absorption and transport of the fluid parts of a cosmetic formulation to the skin. The phrase “starch particles providing solid surfaces” in the cosmetic formulation as used herein means starch in solid particle form having a certain beneficial particle surface related to its size and shape. When solid particles are added to a formulation they add to the solid content in terms of size and weight. In the present invention the importance of the solid surfaces provided to the formulation has been identified. This affects the application of the formulation, the interactions of the cosmetic formulations with skin. The added solid surface contributes to the absorption of other components in the formulation, such as oil, see example 8. Evaporation of other components in the formulation such as water or alcohol can be increased, see examples 9 and 10. Furthermore, small solid particles do not contribute to negative visible or sensory perceived residues on the skin. Fluid parts are generally composed of oil and/or water. Smaller particles contribute to a larger surface per added weight than larger particles since more particles are added per unit weight. This adds to the texture of the cosmetic formulation. Small particles further provide a pleasant surface to the outer layer of the skin. The small size enables the particles to be well distributed to fine wrinkles and furrows found on skin.

Surface area A=4*π*Dₛ² where Dₛ is the particle diameter. The calculation is based on spherical particles.
Volume of sphere V=(4*π*Dₛ³)/6
For starch particles with a solid density ρₛ=1550 kg/m³, and weight of starch, Wₛ, the area is A=(6*Wₛ)/(ρₛ*Dₛ)
i.e., with the same density and weight of starch added, the area added to the cosmetic formulation will depend on the size of the starch particles. Particles of 4 μm and smaller providing solid surfaces contribute to an area of at least 1 m² per g of particles, for instance from 1 to about 20 m² per g particles, e.g. from 1 to 10 m² per g particles, for instance 1 to 5, e.g. 1-3, when added to a formulation, see FIG. 2. For an example size of 0.2 μm the area is 19.35, for an example size of 0.4 μm the area is 9.68, for an example size of 0.5 μm the area is 7.74, for an example size of 0.75 μm the area is 5.16, for an example size of 1 μm the area is 3.87, for an example size of 1.5 μm the area is 2.58, for an example size of 2 μm the area is 1.94, for an example size of 2.5 μm the area is 1.55, for an example size of 3 μm the area is 1.29, for an example size of 3.5 μm the area is 1.11, for an example size of 4 μm the area is 0.97, for an example size of 4.5 μm the area is 0.86, for an example size of 5 μm the area is 0.77.

In an embodiment said starch particles are present in said oil or said starch particles are present in said water or aqueous phase of the cosmetic formulation. The aqueous phase can contain other components including but not limited to humectants, rheology modifiers, alcohols, surfactants, preservatives. The oil may be polar or non-polar and may be chosen from any oil as used in cosmetic formulations including emollients, waxes, essential oils and triglycerides. In another embodiment said starch particles are present in both said water and oil of the cosmetic formulation. The cosmetic formulation provided herein provides texture enhancement by providing transport of oil drops on skin that leads to a non-sticky sensation and feeling of deeper absorbance. The starch based particles in the cosmetic formulation are biodegradable, thus improving the biodegradability of the cosmetic formulation. The starch particles are of natural origin.

In another embodiment said cosmetic formulation is an oil-in-water emulsion or a water-in-oil emulsion and said starch particles are situated at the interface between said oil and water, providing a particle stabilized emulsion. When the starch particles are present at the interface between said oil and water the emulsion is stabilized, thereby reducing phase separation. In another embodiment said starch particles are instead present in said oil or in said water for instance in a case where a surfactant is present to stabilize the emulsion. Another possibility is that said starch particles are present at both said interface between the oil and the water and in said water and/or oil.

In another embodiment, there is provided non-dissolved starch based particles, having a particle size of 0.2-4 μm and having a composition of 0.3-5% by weight of protein, 0.1-4% by weight of lipids, 0-1.6% by weight fibre and <0.45% by weight ash and the remaining part of the composition is carbohydrate adding up to 100% by weight.

In the present context the word “particle stabilized emulsion” is intended to mean an emulsion having at least two phases, wherein the majority of the added starch particles are arranged at the interface between the at least two phases, e.g. at the interface between an oil phase and a water based phase, and thereby stabilizing the emulsion.

Said cosmetic formulation may be chosen from a cream, lotion, toner, gel, serum, primer, foundation, cleanser, deodorant, ointment, oil, body butter, mask, and pigmented product. Said starch particles may be present in said cosmetic formulation in an amount of 1-50% by weight, more specifically 0.1-10% by weight, for instance 0.05-5% by weight, and more specifically 0.05-2% by weight. The amount of starch particles in the formulation may be adapted for the typical application and may also be added in an amount of 0.05, 0.1, 0.2, 0.25, 0.5, 1, 2, 5, 10, 15, 20, 25 or 30% (or what fits the purpose) by weight. Typical amounts are 0.05-5% for texture enhancer and 5-15% as emulsifier (15 if higher oil content is used).

In an embodiment, there is provided a cosmetic formulation comprising starch particles and oil and/or water, wherein said starch particles are present in said oil or said starch particles are present in said water or said starch particles are present in both said water and oil of the cosmetic formulation. The presence of the starch particles in the oil or in the water of the cosmetic formulation provides a unique skin-feel when applied to the skin. In an embodiment of the invention 0.05-2% by weight starch particles, for instance 0.25% by weight of starch particles was detectable.

Said cosmetic formulation may be an oil-in-water emulsion or water-in-oil emulsion and said starch particles are situated at the interface between said oil and water providing a stabilizing effect of the emulsion. Said starch particles may be present in said oil or in said water in a case where a surfactant is present at the interface between the oil and the water. Any surfactant which is suitable for a cosmetic formulation may be used at such instance, e.g. for an oil-in-water emulsions examples are sucrose stearate, glyceryl oleate citrate (non-ionic green), stearyl alcohol, PCA Ethyl cocoyl arginate (cationic), magnesium lauryl sulphate, cera microcristallina (wax); for water in oil emulsions examples are (modified polyether polysiloxane, polyglycerol polyricineoleate). Said starch particles may also be present at said both interface and said water and/or oil.

The composition can be obtained by extraction and removal of components from the botanical origin to reach the composition of protein, lipid, fibre and ash as defined in the claims. Components can also be added to the starch particles to reach the composition of the starch-based particles as defined, for instance added from the original botanical source or from other sources.

In an embodiment, there is provided a process for the preparation of a starch based particle as described above, comprising the following steps:
subjecting dehulled grains or grain press cake to wet or dry milling for providing a flour with the size of 0.2-2.0 mm;
mixing said flour with water for hydration during a period of 0.5-6 hours for efficient fibre separation;
separation of a light phase comprising mainly starch and protein and a heavy phase comprising fibre;
pH adjustment of light phase to pH 6-12;
separation of proteins and starch from light phase based on density and size such as by centrifugal forces; and
drying.

The mixing and separation step as described above may take place in any order, i.e. the mixing may take place before the separation step or the separation step may take place before the mixing step. The pH adjustment step may take place in any order but must take place before the separation of proteins.

The grains may be subjected to common pretreatments such as washing and/or dehulling (with the hull, i.e. the outer shell or coating of the seed removed). Grains may also have been previously subjected to processing to remove liquid content such as lipids, in processes creating a press cake. The grains may be obtained from one of the botanical sources as described herein such as quinoa or amaranth.

The phrase “non-dissolved” as used herein in connection with starch particles means that the starch particles are not soluble, i.e. non-dissolved, in an aqueous phase such as water at room temperature. The non-dissolved particles are furthermore not individual molecules. Molecular starch does dissolve or disintergrate in water and is thus not covered by the wording “non-dissolved” starch particles as used herein. Also terms such as

cold swelling, pre-swelled or pre-gelatinized starch are not covered by the wording “non-dissolved” starch particles. Non-dissolved starch particles are formed by starch molecules, however, the structure and packing of molecules do not allow the starch to be soluble. On the contrary, soluble starch dissolves or disintegrates into molecular form in an aqueous phase such as water at room temperature, i.e. temperatures below 40C.

In the present context the word “starch particles” has the same meaning as starch granules. These phrases may be used interchangeably. Starch particles or starch granules have a size of 0.2-4 μm as used herein and are different from molecular starch having a size of e.g. 110-267 nm.

The invention will be further defined and explained below in the non-limiting examples.

### Experimental Description

### Experiment 1—Quinoa Starch Production

### Pilot Scale Production

The process was performed in small pilot scale starting with 100 kg quinoa grain and designed for further scale up to 2000 kg (quinoa grain start material).

The current process was run for 2 days prior to drying. The total process time can be longer or shorter.

TABLE 1
Quinoa grain/flour composition

| Components | Content wt % |
|---|---|
| Ash | <3 |
| Purity | >99.9 |
| Saponines | <0.02 |
| Protein | 12 |
| Fat | 6 |
| Carbohydrates | 58; sugar 1.5 |
| Dietary Fibers | 7 |
| Moisture content | Max 14 |

### Milling

Dry milling was performed twice with a hammer mill to pass a 1.5 mm sieve. Finer material could result in fine crushed fibers following the starch in the light phase further in the process instead of being separated in the heavy phase during fiber separation. Dry or wet milling can be applied, and different types of mills and sieves can be used as an alternative for production at different scale.

### Mixing—Quinoa Flour Slurry

The quinoa flour was mixed with water, in total 600 L water for 100 kg flour. The slurry was mixed and circulated through a wet mill with 0.45 mm gap. A residence time of 2-4 hours with short runs through the wet-mill for 15 min initially and 5 min every hour have shown reproducible results.

The mixing time allow the starch to release from the fibers prior to the fiber separation. It was desired to avoid having fibers following the light phase, while not losing too much starch in the heavy phase. Mixing can be performed using different instrumentation such as with an eductor, by mixing directly with water in a tank, by using high shear mixing, or a combination of high shear mixing and using a wet-mill, and by allowing longer residence time in water for the starch to separate better from the fibers.

Concentration and other parameter settings have to be adjusted according to the equipment used and the scale of production.

### Fiber Separation

The slurry was mixed by circulation and pumped into decanter 1 with a flow rate of 1.5 m³/h, 1500 rpm. The light phase (LP) mainly containing starch, protein and small fibers, was collected for further processing (dry matter, DM, 5.4 wt %). The heavy phase (HP) mainly contained fiber and some starch aggregates.

The flowrate in this step was 1.5 m³/h and decanter speed 1500 rpm for a volume of 700 L quinoa flour slurry with 12-14 wt % solids.

Wet fibers collected in the heavy phase had a moisture content 62-65 wt %. LP is a liquid dispersion and has a moisture content of 93-95 wt % and 5-7 wt % solids. The HP can be recirculated to increase yield.

Fibre separation can be performed using different methods and equipment such as, but not limited to, sedimentation, centrifuges, centrisieves, hydrocyclons, separators, decanter, sedicanter, sieving and/or combinations of these. The specific parameter settings such as concentrations, flowrate, speed etc, have to be adjusted according to the equipment used and the scale of production.

### Separation/Washing (2 Washes)

The LP passed a 200 μm sieve before being adjusted to pH 9.0 with NaOH during mixing. The slurry was then mixed by circulation and pumped into a decanter with a flow rate of <0.2 m³/h, ca 6000 rpm (ca 3000 g) and high liquid level. The LP mainly contained protein and fiber. The HP, mainly containing starch, was collected.

The HP passed a 200 μm sieve and was diluted with water to DM 5.4 wt % before being pumped into the decanter at similar conditions as the previous separation. The HP was collected and passed a 200 μm sieve before dilution.

For the first starch wash the adjustment of pH was relatively fast. The first separation is usually run with a diluted feed of LP1, usually 5 wt % solids with the main aim to separate starch from protein. The HP in this step usually has 35-40 wt % solids and LP has 3 wt % solids. Most protein is washed away with LP together with fat and remaining fibers.

For the second starch wash, the decanter settings are the same. The concentration of feed was diluted to 3-5 wt % solids. The HP solid content was 35-40 wt % with a 70 wt % yield. The LP solid content was <1 wt % and mainly contained protein and fat that were washed away.

The washing steps can be performed using different methods and equipment such as, but not limited to, sedimentation, centrifuges, crossflow, centrisieves, hydrocyclons, separators, decanter, sedicanter, sieving and/or combinations of these. The specific parameter settings such as concentrations, pH flowrate, speed etc, have to be adjusted according to the equipment used and the scale of production. pH can be adjusted or neutralized before and/or after each step.

### Drying

The starch (55-65 wt % moisture) was air dried in a large heating cabinet by putting the HP in trays and drying at 40° C. After drying the material was milled with a hammer mill to break aggregates formed during the drying process. Other drying techniques could be implemented such as spray drying, fluid bed drying, freeze drying, air drying. The specific parameter settings have to be adjusted according to the equipment used and the scale of production.

### Conclusion to Experiment 1

This process resulted in particles suitable to use in cosmetic formulations to enhance texture and consistency, and provide the formulation with a natural based, renewable ingredient, without causing any odour or negative colour effects.

P3W2 and P5W2 in table 1 were produced according to this protocol. P4W2 was produced according to a similar protocol but did not reach a suitable composition according to claim 1. The main reason was an inadequate mixing before the fiber separation.

P5W1 was produced according to this protocol and was the same batch as P5W2, although only one washing step was performed after the fiber separation and before drying. This resulted in P5W1 not reaching a suitable composition according to claim 1.

### Experiment 2

In experiment 2 the preparation of emulsions by using starch particles with different composition of proteins, lipids, fibre and ash is demonstrated. Table 2 shows in detail the composition and characteristics of the emulsions. The chemical composition is described in % dry basis (db).

### Experimental Procedure

### Emulsification

Emulsions were prepared in glass test tubes, by combining 60% w/w of continuous phase, 28% w/w of dispersed phase and starch particles as defined herein at 12% w/w and emulsified by high shear mixing using Polytron PT 3000 (PT-DA 3007/2, Kinematica Switzerland) at 22000 rpm for 30 s. The starch particles were predispersed in the continuous phase by using vortex mixing (Vibrofix VF1 IKA Laborteknik, Germany) at 2500 rpm for 15 seconds. The dispersed phase was added to this starch and water mixture and vortexed again at 2500 rpm for 15 seconds before being homogenised. The dispersed phase comprised of Medium chain triglyceride oil (caprylic/capric triglyceride, Tricaprylin) and the continuous phase comprised of distilled water.

### Particle Size Measurements of Starch Granules and Emulsions

The particle size distributions (PSD) were measured after emulsification using laser diffraction (Mastersizer S, Malvern, Worcestershire UK) for starch and starch covered emulsions, and the refractive index of 1.54 was used. A small volume of sample was added to the flow system and pumped through the optical chamber for measurements.

### Microscopy

The emulsions were diluted 30 times with the continuous phase and a drop of the sample was smeared on glass slide without cover to be viewed under light microscope (Leica DMRE, upright light microscope, Leica microsystems, Germany). Microscopy images of the emulsions were obtained using a digital camera attached to the microscope (Leica DFC 500, Leica microsystems, Germany)

### Chemical Analysis

Protein content was analysed according to method equivalent to ISO 16634-1:2008 (Dumas method) except PEX13 that was analysed according to NMKL 6:4, 2003 (Kjeldhal method). Lipid content were analysed according to method equivalent to 2009/152/EU except PEX13 that was analysed according to NMKL 131, 1989. Fibre content was analysed according to method equivalent to 2009/152/EU. Ash content was analysed according to 2009/152/EU.

### Physical Characteristics

After emulsification, the colour of samples was evaluated by the eye, and the odour by smell. Negative effects for these properties could be directly related to the starch particles added. In an additional test starch was treated at 150° C. for 120 min before emulsification and the odour of emulsions evaluated. This treatment enhanced the negative odour further when present.

### Free Starch

The amount of free starch has been calculated using the equation below.

Free starch=Volume % free starch÷Volume % emulsion

The Volume % free starch is the height of the PSD curve peak for free starch (i.e. with the same size as the starch particles)

Volume % emulsion is the height of the PSD curve peak for the emulsion (with larger size than the starch particles) in the same PSD curve.

### Conclusion to Experiment 2

From Table 2 it can be seen that a starch particle having a certain composition of protein, fibre and lipids provides an emulsion with desired characteristics to be used in a cosmetic formulation. Effects on odour and colour are not limited to emulsion formulations. A homogenous consistency is important for all types of cosmetic formulations. From Table 2 it is clear that samples PEX13, L1602, P2W3, P3W2, P5W2 have a composition providing desired results for all these parameters. These samples also have a value of the free starch <0.45. A low value of free starch is preferable. Samples PEX15, P4W1, P4W2, P5W1 have non-desired results for more than one parameter. These four samples all gave non-homogenous consistency, more free starch, and negative odour after heating. PEX15, P4W1, P4W2 gave off-white or yellowish colour, and odour was detected in PEX15, P4W1 & P5W1 before heating. Thus, in view of the above it is clear that it is important to keep the composition of the starch particles within the claimed ranges in order to provide an emulsion with acceptable odour and colour and beneficial consistency.

TABLE 2

| Quinoa | | | | | | | | Odour | Pickering | |
|---|---|---|---|---|---|---|---|---|---|---|
| starch | Protein | Lipids | Fibre | Ash | Free | | Initial | after | emulsion | Emulsion |
| sample | (% db) | (% db) | (% db) | (% db) | starch^{a} | Colour | Odour | heating | (Y/N) | consistency |
| PEX13 | 0.99 | 3.74 | 0.1^{b} | 0.44 | 0.29 | white | N | N | Y | Homogenous |
| L1602 | 3.8 | 1.43 | 0.3^{b} | 0.2 | 0.17 | whitish | N | N | Y | Homogenous |
| PEX15 | 7.2 | 2.92 | 3.3 | 1 | 0.47 | yellowish | Y flour | Y flour | Y | Grainy |
| P2W3 | 1.30 | 0.46 | 1.0^{b} | 0.1^{b} | 0.16 | whitish | N | N | Y | Homogenous |
| P3W2 | 0.83 | 0.51 | 1.0^{b} | 0.22 | 0.19 | whitish | N | N | Y | Homogenous |
| P4W1 | 2.55 | 1.01 | 2.23 | 0.71 | 0.7 | off-white | Y flour | Y flour | N | Low in viscosity |
| P4W2 | 1.31 | 0.40 | 2.32 | 0.49 | 0.54 | off-white | N | Y flour | N | Inhomogeneous |
| P5W1 | 2.30 | 2.0^{b} | 1.0^{b} | 0.48 | 0.49 | whitish | Y | Y | Y | Inhomogeneous |
| | | | | | | | | cookie | | |
| P5W2 | 0.97 | 0.20 | 1.0^{b} | 0.17 | 0.43 | whitish | N^{c} | N | Y | Homogenous |

The conclusion is that it is only certain starch particles that are especially suited for being used in cosmetic formulations. In the table ^{a}, ^{b}, and ^{c} are disclosed meaning ^{a} volume % free starch/Volume % emulsion, ^{b}Less than, ^{c}a slight odour of starch may be detected but not a strong odour or flour odour.

A starch particle having a combination of protein, fibre and lipids suitable for cosmetic formulations can be clearly limited in a three-dimensional space. This can be illustrated in two dimensions by FIG. 3 and FIG. 4, showing the protein content in relation to the fibre content, and the lipid content in relation to the fibre content, respectively. The borders of these limitations can be described by the equations below. The composition of the starch based particles should be within the limitation of both equations and the total composition according to claim 1.

| | |
|---|---|
| Protein ≤ | 1.7/Fibre |
| Lipid ≤ | −3 * Fibre + 4.5 |

The amount of protein corresponds to an amount equal to or less than 1.7 divided by the amount of fibre in weight percent divided by 100. The amount lipid is equal to or less than −3 multiplied by the amount of fibre in weight percent divided by 100 followed by the addition of 4.5. Experiment 3

In experiment 3 dispersions and emulsions representing cosmetic formulations were created using starch particles to demonstrate the effect on skin.

### Experimental Procedure

Starch was added to a medium such as oil (caprylic/capric triglyceride), water (deioinised water), silicones (Dimethicone) and water thickened with a rheology modifier (Carbomer). The starch particles were dispersed using high shear mixing using Polytron PT 3000 (PT-DA 3007/2, Kinematica Switzerland) at 22000 rpm for 30 s. The amount of starch particles used was in the range of 0.25% to 10% to demonstrate presence of particles in the water phase, oil phase or on the interface between oil and water.

For each test a reference sample was created without addition of any starch.

The physically modified quinoa starch particles used in the experiment were from sample W2P5 from Table 2 with further treatment. Physical modification was performed by dry thermal treatment of the starch particles at 150° C. for 120 min. The amaranth starch sample was physically treated by the same method as the quinoa starch. The chemically modified quinoa starch sample was modified with 2.5% OSA and washed with water after modification. The composition of the chemically treated sample was 0.8% protein, 0.2% lipids, 1% fibre, and 0.6% ash measured as in example 2.

The quinoa starch particles used in the experiment were from sample W2P5 from Table 2 with further treatment. Physical modification was performed by dry thermal treatment of the starch particles at 150 C for 120 min. The chemically modification sample was modified with 2.5% OSA.

For emulsions with starch present in water or oil but not at the interface, 28% (w/w) O/W emulsions were prepared using a non-ionic surfactant (PEG-6 Stearate (and) Ceteth-20 (and) Glyceryl Stearate (and) Steareth-20) at 5% w/w with the remaining 67% with distilled water. Whereas, for emulsions with starch at the oil-water interface no surfactant was used.

A sensory analysis was performed by two people. Sensory parameters were evaluated from a small amount of the emulsion being applied on the forearm and participants grading the samples on presence or absence of particles. For further understanding see Table 3 which shows the on-skin particle detection test in dispersions, emulsions and mediums with details regarding source, amount and position of the particles.

TABLE 3

| | | | | Position of | |
|---|---|---|---|---|---|
| | | Amount of | | particles | Detection of |
| Source of | | particles | | in cosmetic | particles on |
| Particles | Modification | (% w/w) | Dispersion/Emulsion/Medium | formulation | skin |
| Quinoa | Physical | 0.25 | Oil dispersion | Oil phase | Yes |
| Quinoa | Physical | 0.5 | Oil dispersion | Oil phase | Yes |
| Quinoa | Chemical | 0.5 | Oil dispersion | Oil phase | Yes |
| Reference | — | 0 | Oil | — | No |
| Amaranth | Physical | 0.5 | Oil dispersion | Oil phase | Yes |
| Quinoa | Physical | 0.5 | Silicone (phase) | Silicone phase | Yes |
| | | | dispersion | | |
| Reference | — | 0 | Silicone | — | No |
| Quinoa | Physical | 0.5 | Aqueous dispersion | Water phase | Yes |
| Reference | — | 0 | Aqueous | | |
| Quinoa | Physical | 0.5 | Aqueous dispersion with | Water phase | Yes |
| | | | rheology modifier | | |
| Reference | — | 0 | Aqueous with rheology | — | No |
| | | | modifier | | |
| Quinoa | Physical | 0.5 | O/W Emulsion | Oil phase | Yes |
| Quinoa | Physical | 0.5 | O/W Emulsion | Water phase | Yes |
| Reference | — | 0 | O/W Emulsion | — | No |
| Quinoa | Physical | 10 | O/W Emulsion | Oil phase & | Yes |
| | | | | interface | |
| Quinoa | Physical | 10 | O/W Emulsion | Water phase & | Yes |
| | | | | interface | |
| Reference | — | 0 | Oil and Water | — | No |

### Conclusions from Experiment 3

The texture benefit of the particles were detected on skin in all samples. The participants were clearly able to differentiate between presence or absence of starch particles when they are in different phases of a dispersion or emulsion.

### Experiment 4

In experiment 4, a correlation is shown between hydrophobicity of the particles and amount of free starch which can affect the interaction of particles with skin.

### Experimental Procedure

Quinoa starch particles were analysed without further treatment, after physical modification, and after chemical modification, respectively, as described in experiment 3. O/W emulsions were made according to the method described in experiment 2. This was performed as a way to determine free starch according to the following equation

Hydrophobicity∝1+Free starch

as a measure of the hydrophobicity of starch particles. Free starch was calculated according to experiment 2. For further understanding see Table 4 which shows the amount of free starch in modified and non-modified starch particles.

TABLE 4

| Quinoa | | |
|---|---|---|
| starch | | |
| sample | Modification | Free starch |
| P3W2 | Non-modified | 0.19 |
| P3W2 | Physically modified | 0.11 |
| P3W3 | Chemically modified | 0.04 |
| P5W2 | Non-modified | 0.43 |
| P5W2 | Physically modified | 0.17 |
| P5W3 | Chemically modified | 0.05 |

### Conclusion to Experiment 4

Charges and hydrophobicity of the particles can affect the interaction with skin lipids and distribution of lipids in the cosmetic formulation to the skin. The free starch in Table 4 is an indication of the hydrophobicity of the starch particles. The hydrophobicity is influenced by the composition and processing of the starch particles. For starch produced according to Experiment 2, free starch for desired samples was <0.45. With a thermal heat treatment (in this example 120° C. for 150 minutes), the proteins at the surface were further hydrophobized increasing the oil binding ability of the starch and reducing the free starch. For P3W2 from 0.19 to 0.11, and for P5W2 from 0.43 to 0.17. Chemical modification also reduced the free starch. This indicates the potential of the starch particles to interact with skin lipids.

### Experiment 5

In experiment 5, a hand cream based on an O/W emulsion was formulated with commonly used ingredients for cosmetic formulations and starch particles corresponding to P3W2 were added.

### Experimental Procedure

The ingredients used in the formulation and the composition is listed in Table 5. Phase A (aqueous phase) and phase B (oil phase) were prepared separately and heated to 70° C. Phase A and B were then mixed together and emulsified with IKA T25 Ultra Turrax at 15000 rpm for 2 minutes. The system was then mixed during cooling down until the temperature was below 50° C. Phase C (glycerine and quinoa starch particles) was then added and the formulation thoroughly mixed.

The surface (A) added to the formulation by including starch particles was calculated per ml of formulation as A=(6*Ws)/(ρs*Dₛ). Ws was calculated as 1% starch per 1 ml of formulation, equal to 0.01 g or 0.00001 kg, the solid density ρs=1550 kg/m³ and particle diameter Dₛ=1.5*10-6 m. The added surface was 0.026 m2/ml formulation.

TABLE 5

| Phase | Ingredients | Function | Content w/w (%) |
|---|---|---|---|
| A | Deionized Water | Aqua | Up to 100 |
| A | Phenoxyethanol | Preservative | 1.00 |
| A | Caprylyl Glycol | Preservative | 0.50 |
| B | Glyceryl stearate citrate | Surfactant | 3.00 |
| B | Cetearyl alcohol | Surfactant | 5.00 |
| B | PEG-100 stearate | Surfactant | 1.00 |
| B | Dimethicone | Emollient | 2.0 |
| B | Isopropyl palmitate | Emollient | 6.0 |
| B | Canola oil | Emollient | 2.0 |
| B | Caprylic/capric triglyceride | Emollient | 8.00 |
| C | Quinoa starch particles | Quinoa Starch | 1.00 |
| C | Glycerin | Glycerin | 3.00 |
| | | | 100.00 |

### Conclusion from Experiment 5

The starch particles were homogeneously dispersed in the formulation providing a good oil absorption and a pleasant texture to the skin after application.

### Experiment 6

In Experiment 6 the effect of adding small particles even at a low dosage was shown. Small starch particles can improve the skin feel of formulations even in very small percentages. Adding a minimum quantity of the particles to a formulation after the emulsification process provides a new texture that can be appreciated as an improvement by end-users.

Five creams were formulated following the same recipe but with addition of different percentages of starch particles after the emulsification process. The samples were then evaluated by a panel including 16 different persons.

### Experimental Procedure

The ingredients used in the formulation and the composition is listed in Table 6 (“Composition”). Phase A (aqueous phase) and phase B (oil phase) were prepared separately and heated to 70° C. and 50° C. respectively. Phase A and B were then mixed together at 50° C. and emulsified with an IKA TN25 Ultra Turrax (S25N 10G dispersion tool) at 15 000 rpm for 2 minutes. The system was then mixed during cooling down until the temperature was below 50° C. Phase C (glycerine and starch particles, L1602 from Experiment 2 as seen in Table 2) was added to the formulation after cooling down, and thoroughly mixed using an overhead stirrer. Five different samples were prepared, each with a different percentage of starch (0%, 0.05%, 0.1%, 2% and 5%).

### Sensory Analysis

The five samples were evaluated by a panel including sixteen persons, of which 12 were female and 4 male. The panelists were requested to select the most preferred sample. Samples were presented to the panel by a randomised number and in random order.

### Conclusion to Experiment 6

The conclusion from experiment 6 was that small starch particles improved the overall perceived texture of a cream. The effect was detectable even at a very low dosage (0.05%). 12.5% of the panel selected the sample with 0.05% of particles as the preferred one. The most preferred sample was the cream with 2% starch, selected by 43.75%. None of the panelists (0%) preferred the reference sample without starch particles. These results are shown in Table 6.

TABLE 6
Composition of samples prepared for experiment 6.

| | | | Sample | Sample | Sample | Sample | Sample |
|---|---|---|---|---|---|---|---|
| | | | 0% | 0.05% | 0.1% | 2% | 5% |
| Phase | Ingredient | Function | Starch | Starch | Starch | Starch | Starch |
| A | Deonized water | Aqua | Up to 100 | Up to 100 | Up to 100 | Up to 100 | Up to 100 |
| A | Phenoxyethanol | Preservative | 1.00 | 1.00 | 1.00 | 1.00 | 1.00 |
| A | Caprylyl Glycol | Preservative | 0.50 | 0.50 | 0.50 | 0.50 | 0.50 |
| B | Glyceryl | Surfactant | 3.00 | 3.00 | 3.00 | 3.00 | 3.00 |
| | stearate | | | | | | |
| B | Cetearyl alcohol | Surfactant | 5.00 | 5.00 | 5.00 | 5.00 | 5.00 |
| B | PEG-100 | Surfactant | 1.00 | 1.00 | 1.00 | 1.00 | 1.00 |
| | stearate | | | | | | |
| B | Isopropyl | Emollient | 6.00 | 6.00 | 6.00 | 6.00 | 6.00 |
| | palmitate | | | | | | |
| B | Canola oil | Emollient | 2.00 | 2.00 | 2.00 | 2.00 | 2.00 |
| B | Caprylic/capric | Emollient | 8.00 | 8.00 | 8.00 | 8.00 | 8.00 |
| | triglyceride | | | | | | |
| C | Glycerin | Humectant | 3.00 | 3.00 | 3.00 | 3.00 | 3.00 |

TABLE 7
Preferred sample results from sensory panel test in experiment 6.

| | |
|---|---|
| Sample | % of panel selecting sample |

| | | |
|---|---|---|
| 0% | Starch | 0% |
| 0.05% | Starch | 12.5% |
| 0.1% | Starch | 18.75% |
| 2% | Starch | 43.75% |
| 5% | Starch | 25% |

### Experiment 7

In Experiment 7 an effect of small starch particles comparable to silicones in cream formulations was evaluated. Adding 1% of small starch particles to a formulation after the emulsification process provided a texture comparable to the one provided by silicones, and which was not achieved by adding larger starch particles.

Four creams were formulated following the same basic recipe, although with differently sized starch particles added, or with small starch particles and no Dimethicone in the formulation. Dimethicone is commonly used to improve the sensorial attributes of formulations, to increase the ease of spread of the formulation over the skin, and conditioning and protection of skin. The samples were evaluated by a panel including 15 persons.

### Experimental Procedure

The ingredients used in the formulation and the composition of each formulation is listed in Table 8. Phase A (aqueous phase) and phase B (oil phase) were prepared separately and heated to 70° C. and 50° C. respectively. Phase A and B were then mixed together at 50° C. and emulsified with IKA TN25 Ultra Turrax at 15000 rpm for 2 minutes. The system was then mixed during cooling down until the temperature was below 50° C. Phase C (glycerine and starch particles) was then added and the formulation thoroughly mixed with an overhead stirrer. Four different samples were prepared, i.e. with silicone (Dimethicone, BRB 350 IMCD) and without any starch particles (D+NS), with silicone and 1% small starch particles (D+S), with 1% small starch and without silicone (ND+S), with silicone and 1% large starch particles (D+XS), respectively. The small starch particles, 1.54 μm, were L1602 from Experiment 2 as seen in Table 2. The large starch particles, 14.97 μm, were corn starch (Zea Mays Starch, Organic Makers).

### Sensory Analysis

The four samples were evaluated by a panel including 15 persons, of which were 11 female and 4 male. Samples were presented to the panel by a randomised number and in random order. The panelists ranked attributes before and during application, after-feel, and were asked to select their preferred sample.

### Conclusion to Experiment 7

The conclusion from Experiment 7 was that the samples containing Dimethicone and/or quinoa starch particles were extremely similar, 27% of the panel actively noted that samples were hard to rank in comments. Therefore, most sensorial attributes where not significantly different. When selecting their preferred samples, there was no difference between the sample with dimethicone only, or together with small starch particles (33.3% selected each), i.e. panelists preferred a silicone feel, with or without small starch particles. The sample with small starch particles but without silicone was, however, preferred over the silicone sample including large starch particles (only preferred by 13.3%). Therefore it was clear that large starch particles reduced the sensorial attributes of the cream, whereas the small particles provided attractive sensorial attributes.

TABLE 8
Composition of samples prepared for experiment 7.

| Phase | Ingredient | Function | D + NS | D + S | ND + S | D + XS |
|---|---|---|---|---|---|---|
| A | Deonized water | Aqua | Up to 100 | Up to 100 | Up to 100 | Up to 100 |
| A | Phenoxyethanol | Preservative | 1.00 | 1.00 | 1.00 | 1.00 |
| A | Caprylyl Glycol | Preservative | 0.50 | 0.50 | 0.50 | 0.50 |
| B | Gliceryl stearate | Surfactant | 3.00 | 3.00 | 3.00 | 3.00 |
| B | Cetearyl alcohol | Surfactant | 5.00 | 5.00 | 5.00 | 5.00 |
| B | PEG-100 stearate | Surfactant | 1.00 | 1.00 | 1.00 | 1.00 |
| B | Dimethicone | Emollient | 2.00 | 2.00 | 0.00 | 2.00 |
| B | Isopropyl palmitate | Emollient | 6.00 | 6.00 | 6.00 | 6.00 |
| B | Canola oil | Emollient | 2.00 | 2.00 | 2.00 | 2.00 |
| B | Caprylic/capric triglyceride | Emollient | 8.00 | 8.00 | 8.00 | 8.00 |
| C | Glycerin | Humectant | 3.00 | 3.00 | 3.00 | 3.00 |
| C | Quinoa starch particles | Texturizer | 0.00 | 1.00 | 1.00 | 0.00 |
| C | Corn starch particles | Texturizer | 0.00 | 0.00 | 0.00 | 1.00 |

TABLE 8A
Preferred sample in experiment 7.

| Sample | Starch diameter (μm) | Preferred by (%) |
|---|---|---|
| D + NS | Not present | 33.3 |
| D + S | 1.54 | 33.3 |
| ND + S | 1.54 | 20.0 |
| D + XS | 14.97 | 13.3 |

### Experiment 8

In Experiment 8, the same amount of different starches have been added to oil and then a drop of each sample was deposited on absorbent paper to evaluate the oil absorption. Oil absorption is an important feature of a cosmetic formulation, affecting the delivery of emollients (oils) into the skin.

### Experimental Procedure

One gram of different native starches, from Quinoa, Corn (as in Experiment 7), Tapioca (La Carla), and Rice (Sigma-Aldrich), respectively, was added to five grams of jojoba oil (NaturaTec) and mixed by hand during one minute in a 25 ml beaker until being homogenously dispersed. The composition of each sample was 17% starch and 83% oil. The relation between particle diameter and particle area per 1 g of starch is seen in FIG. 2.

Thereafter, 50 μl of each sample has been deposited on a Munktell Retention 2 filter paper using a Gilson micropipette. After five minutes, measured with a chronometer, the diameter of the oil on the paper was measured with a ruler, the obtained results can be seen in Table 9. Good absorption of the sample applied would result in a small diameter, whereas lower absorption of the oil would result in the oil spreading to a larger area of the filter paper.

### Conclusion to Experiment 8

The area covered by the jojoba oil was smaller when adding the smallest starch, quinoa, than for the other starches. The small starch particles thereby were seen to enhance the oil absorption more efficiently than larger starches. This would be expected due to the larger solid surface of the small particles. The solid surface would aid the delivery of oil to the upper skin layers, allowing the oil to transport along the particle surfaces. An efficient oil absorption further can decrease the greasy feel of a formulation, thus improving both sensory attributes and the functionality of the cosmetic formulation on skin.

TABLE 9
Oil absorption measured as the time spread of oil by diameter for
samples with differently sized starch particles for Experiment 8.

| | | Oil sample |
|---|---|---|
| Sample | Starch diameter (μm) | diameter (cm) |
| Quinoa Starch + Jojoba oil | 1.54 | 1.8 |
| Rice Starch + Jojoba oil | 5.47 | 2.1 |
| Tapioca Starch + Jojoba oil | 14.91 | 2.4 |
| Corn Starch + Jojoba oil | 14.97 | 2.3 |

### Experiment 9

Evaporation of excess water occur from aqueous formulations. An enhanced evaporation can improve the delivery and sensory feel of other, non evaporated, components such as emollients and actives. A larger solid surface area per amount of particles is expected to enhance evaporation of excess liquid. Therefore, starch particles of different size when dispersed in water would affect the evaporation, i.e. in different evaporation times for the same amount of particles.

In Experiment 9 different starches have been dispersed in water in the same percentages and then were deposited on petri dishes and let to evaporate. The time for water evaporation for all samples was then evaluated.

### Experimental Procedure

One gram of different native starches (from Amaranth (Biopolymer), and Quinoa, Rice, Tapioca, and Corn as in Experiment 8) was added to eight grams of water and mixed until being well dispersed in a 25 ml beaker.

Then, 100μ of each sample was placed on a petri dish using a Gilson micropipette. The time for water evaporation for each sample was followed using a chronometer and visual observation. The experiment was done in the laboratory at room temperature (22° C.). The samples were considered evaporated when no free water in the sample was observed and there was only starch left, the obtained results can be seen in Table 10.

### Conclusion to Experiment 9

Smaller starch particles such as Amaranth starch particles and Quinoa starch particles increased the evaporation velocity. The evaporation from the samples containing Tapioca, Corn and Rice starches were much slower compared with the samples containing Amaranth or Quinoa. Thereby the larger solid surface of the these particles had a significant impact on the evaporation behavior. Thereby the smaller starch particles have ability to improve evaporation related delivery and sensory feel of cosmetic formulations.

TABLE 10
Evaporation time for samples with differently
sized starch particles for Experiment 9.

| Sample | Starch diameter (μm) | Time (min) |
|---|---|---|
| Amaranth starch + water | 1.49 | 20 |
| Quinoa Starch + water | 1.54 | 25 |
| Rice Starch + water | 5.47 | 46 |
| Tapioca Starch + water | 14.91 | 58 |
| Corn Starch + water | 14.97 | 42 |

### Experiment 10

In Experiment 10, the enhanced evaporation of an aqueous phase caused by particles with large solid surface was further quantified. The effect of the larger solid surface was evaluated by following weight of water evaporated with time during 1 hour.

### Experimental Procedure

One gram of different starches (as used in Experiment 9) was added to eight grams of water and mixed until being well dispersed in a 25 ml beaker. Samples were mixed with vortex (IKA Vibrofix VF1 Electronic) for 15 seconds and then with IKA Ultra turrax TN 25 with S25N 10G dispersion tool for 30 seconds. 100μ of each sample was placed in a weighing boat using a Gilson micropipette. The evaporation of water was then followed by weight loss using a Mettler-Toledo balance (model HR73) and measuring every fifth minute during one hour. The experiment was done in the laboratory at room temperature (22° C.). The results are seen in Figure NBR? And ??. The relative evaporation rate was evaluated as the measured sample weight (g) divided by the initial sample weight (g initial weight) of the specific sample. The evaporation by total solid surface area was evaluated as the evaporation weight loss divided by the surface area of the sample. The surface area was determined based on particle size, surface and concentration. Triplicate samples were prepared and analyzed for all starches.

### Conclusion to Experiment 10

Smaller starches particles such Amaranth starch and Quinoa starch significantly increased the evaporation of excess liquid compared to larger starch particles. Furthermore the difference between the smallest starches, amaranth and quinoa, and rice starch was larger than the difference between rice starch and the larger starches, tapioca and corn. This despite that the rice starch particle diameter is closer the smaller starches, showing the importance of the solid surface for the evaporation behaviour. The increased evaporation from formulations containing small starch particles may therefore be used to improve cosmetic formulations where functions related evaporation are important, such as for delivery of emollients and actives, and for sensory behaviour.

1-14. (canceled)

15. A cosmetic formulation comprising
(a) starch based particles having a particle size of 0.2-4 μm, said starch based particles having a composition of 0.3-5% by weight of protein, 0.1-4% by weight of lipids, 0-1.6% by weight fiber and <0.45% by weight ash and the remaining part of the composition is carbohydrate adding up to 100% by weight; and
(b) oil, water, or a combination thereof.

16. The cosmetic formulation of claim 15, wherein said starch based particles have a composition of 0.4-2% by weight of protein, 0.1-1% by weight of lipids, 0.1-1.2% by weight fiber and <0.25% by weight ash and the remaining part of the composition is carbohydrate adding up to 100% by weight.

17. The cosmetic formulation of claim 15, wherein said starch based particles are within the limitation of the equations Protein≤1.7/Fiber and Lipid≤−3*Fiber+4.5.

18. The cosmetic formulation of claim 15, wherein said starch based particles are non-gelatinized or native or technically or chemically modified.

19. The cosmetic formulation of claim 15, wherein said particles are from a botanical source chosen from quinoa, amaranth, tapioca, rice, oat, wheat, barley, millet, canihua, including waxy and high amylose varieties of any of the previously mentioned botanical sources.

20. The cosmetic formulation of claim 15, wherein said starch based particles provide solid surfaces in the cosmetic formulation.

21. The cosmetic formulation of claim 20, wherein the solid surfaces contribute to an area of at least 1 m² per g of particles when added to the cosmetic formulation.

22. The cosmetic formulation of claim 15, wherein said cosmetic formulation is an oil-in-water emulsion or a water-in-oil emulsion and said starch based particles are:
(i) situated at the interface between said oil and water,
(ii) present in said oil or in said water, or
(iii) present at said interface and in said water or oil.

23. The cosmetic formulation of claim 15, wherein said cosmetic formulation is selected from a cream, lotion, toner, gel, serum, primer, cleanser, foundation, deodorant, ointment, oil, body butter, mask, and pigmented product.

24. The cosmetic formulation of claim 15, wherein said starch based particles are present in said cosmetic formulation in an amount of 1-50% by weight of the cosmetic formulation.

25. The cosmetic formulation of claim 15, wherein said starch based particles are present in said cosmetic formulation in an amount of 0.1-10% by weight of the cosmetic formulation.

26. The cosmetic formulation of claim 15, wherein said starch based particles are present in said cosmetic formulation in an amount of 0.05-5% by weight of the cosmetic formulation.

27. The cosmetic formulation of claim 15, wherein said starch based particles are present in said cosmetic formulation in an amount of 0.05-2% by weight of the cosmetic formulation.

28. A process for preparing the starch based particles of claim 15, comprising:
(a) subjecting dehulled grains or grain press cake to wet or dry milling to provide a flour with the size of 0.2-2.0 mm;
(b) mixing said flour with water for a period of 0.5-6 hours to release starch from fibers of the flour;
(c) separating a light phase comprising starch and protein from the fiber;
(d) adjusting the pH of the light phase to pH 6-12;
(e) separating proteins from the starch of the light phase based on density and size; and
(f) drying the starch to produce starch based particles.
